(19) United States
(12) Patent Application Publication
(10) Pub. No.: US 2020/0397822 A1
(43) Pub. Date: Dec. 24, 2020

(54) PERSONALIZED TUMOR ANTIGEN SPECIFIC T CELL LINES FOR CANCER TREATMENT

(71) Applicant: Jinbo Liu, San Francisco, CA (US)

(72) Inventor: Jinbo Liu, San Francisco, CA (US)

(21) Appl. No.: 16/576,725

(22) Filed: Sep. 19, 2019

Related U.S. Application Data
(60) Provisional application No. 62/921,460, filed on Jun. 18, 2019.

(51) Int. Cl. A61K 35/17; C12N 5/0783

(52) U.S. Cl. CPC A61K 35/17; C12N 2501/998; C12N 2501/2302; C12N 5/0636

(57) ABSTRACT

The present invention relates to the immunotherapy of cancer. The present invention relates to develop personalized tumor antigen specific T cell lines to treat all types of cancer. The tumor antigen specific T cell lines provide potent and abundant tumor antigen specific T cells that selectively recognize cancer cells and destroy the cancer cells to cure cancer. Personalized tumor antigen specific T cell lines are a safe and effective immunotherapy for cancer treatment. It provides a promise that cancer will be no longer an incurable disease.

### CROSS REFERENCE TO RELATED APPLICATION

This application claims the benefit of U.S. Provisional Patent Application Ser. No. 62/921,460, filed Jun. 18, 2019, the content of which is incorporated herein by reference in its entirety.

### FIELD OF INVENTION

The present invention relates to a method of treating cancer using immunotherapy. In particular, the present invention relates to tumor antigen specific T cell lines for use in cancer treatment. The present invention relates to personalized tumor antigen specific T cell lines to treat all types of cancer.

### BACKGROUND OF INVENTION

Cancer remain incurable so far. Especially for solid cancer, surgical removal is still a primary therapy but is usually curative only when the cancer is diagnosed at its early stages. However, by the time cancer become symptomatic, the best chance for a surgical cure is usually past. Consequently, cancer is one of the leading cause of death in the worldwide.

Immunotherapy appears to be a promising approach for cancer treatment. However, FDA approved immunotherapy drugs or immunotherapy drugs that are in clinical trials (e.g. antibodies, antibody-drug conjugates, cytokines, CAR-T) do not work for all types of cancer. In addition, those drugs develop anti-drug antibodies to diminish their efficacy, develop severe side effects or suppress patient own immunity. CAR-T cells show promise but cause severe side effects such as cytokine storm syndrome and neurotoxicity.

Andrea Mahr et al from IMMATICS BIOTECHNOLOGIES GMBH (Tuebingen, DE) obtained more than 90 patents about tumor associated antigen (TAA) identification within last a few years. However, the tumor associated antigens identified by his team were from a group of patients' tumors and a group of healthy donors' tissues. As we know, the protein or peptide expression on tumor cells from different types of cancer or the same type of cancer from different individuals is not always the same. We have to treat each cancer patient individually in most of cases. There are no universal tumor specific or associated antigens for all patients who have the same type of cancer or different types of cancer. In addition, tumor associated antigen T cells may induce potential harmful immunogenicity to normal cells because tumor associated antigens are expressed on normal cells.

First FDA-approved therapeutic cancer vaccine Provenge (Sipuleucel-T) for prostate cancer is autologous prostate acid phosphatase (PAP)-specific T cells, and prolonged median survival by 4 months compared to placebo (Martin et al. Clinical Cancer Research, 17: 3520-6, 2011). Tumor-reactive T cells generated from melanoma patients' peripheral blood mononuclear cells (PBMC) stimulated with irradiated autologous tumor cells for four weeks. The melanoma patients treated with the tumor-reactive T cells showed impressive results. The months of patients' survival were consistent with the numbers of infused tumor reactive T cells (Verdegaal et al. Cancer Immunol Immunotherapy, 60:953-963, 2011). Tumor antigen specific T cells for cancer treatment appear more and more important because of less side effects. However, how to produce potent and abundant tumor antigen specific T cells remains challenging but is critical to the outcomes of cancer treatment.

### SUMMARY OF INVENTION

The present invention provides a cancer immunotherapy comprising using personalized tumor antigen specific T cell lines to treat all types of cancer.

One aspect of present invention comprises a method of generating tumor antigen specific T cell lines that provide potent and abundant tumor antigen specific T cells for cancer treatment. The tumor antigen specific T cell lines are able to maintain in a cell culture incubator for two years or longer without loss of anti-tumor immunity. In addition, the tumor antigen specific T cell lines can be frozen and stored in a liquid nitrogen freezer, thawed and expanded in a cell culture incubator as needed.

Another aspect of the present invention is to identify tumor specific antigens (e.g. proteins, peptides) from each individual cancer patient and develop a personalized tumor specific antigen T cell line to treat a cancer patient.

Another aspect of present invention further comprises tumor antigen specific T cell lines are developed and expended in vitro using a cancer patient's own peripheral blood mononuclear cells (PBMC) or an identified healthy donor PBMC. The tumor antigen specific T cell line that shows strong anti-tumor immune responses is selected to treat the cancer patient.

Another aspect of present invention furthermore comprises that a tumor antigen specific T cell line developed from a cancer patient shows strong anti-tumor immune responses to another cancer patient's tumor cells and the tumor antigen specific T cell line may be selected to treat the another cancer patient.

### BRIEF DESCRIPTION OF THE DRAWINGS

FIG. 1 is a process flow diagram illustrating a method for developing autologous tumor antigen specific T cell lines to treat a cancer patient using tumor specific antigens (e.g. proteins and/or peptides) identified from the cancer patient.

FIG. 2 is a process flow diagram illustrating a method for developing an autologous tumor antigen specific T cell line to treat a cancer patient using mitomycin-treated tumor cells from the cancer patient.

FIG. 3 is a process flow diagram illustrating a method for treating a cancer patient using an allogeneic tumor antigen specific T cell line that is developed from another cancer patient.

FIG. 4 is a process flow diagram illustrating a method for developing allogeneic tumor antigen specific T cell lines using a healthy donor PBMC stimulated with identified tumor specific antigens (e.g. proteins and/or peptides) from a cancer patient. The tumor antigen specific T cell line that has strong anti-tumor immune responses to the tumor specific antigens or tumor cells from the cancer patient will be selected to treat the cancer patient.

FIG. 5 is a brief drawing of how a tumor antigen specific T cell line to kill cancer cells.

FIG. 6 is an example of the T cell immune response from an antigen specific T cell line. IFN-gamma producing cells were increased 10 to 100 times upon the antigen re-stimulation.

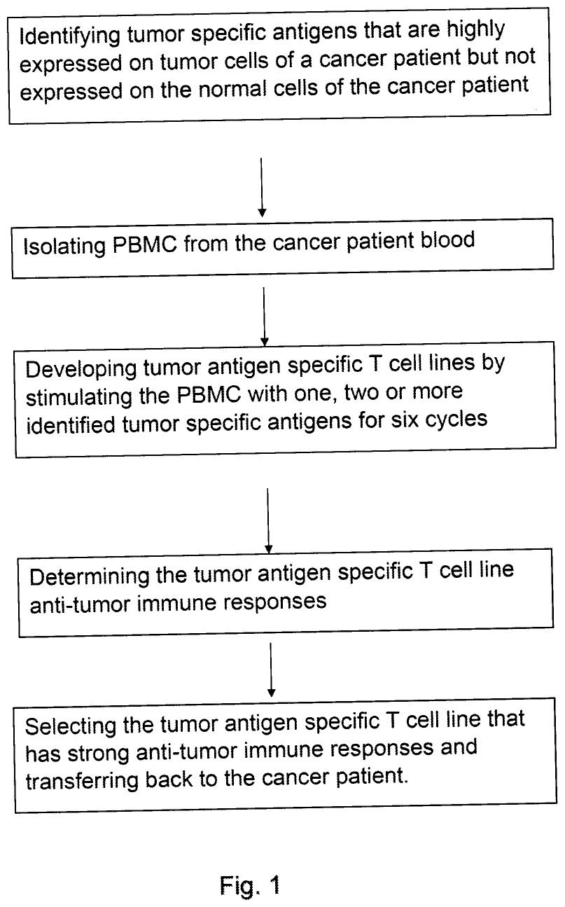

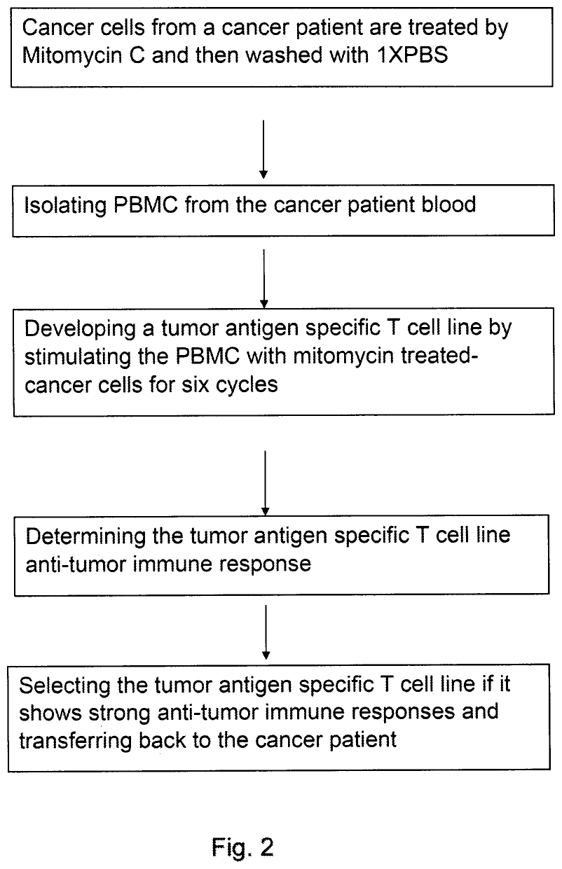

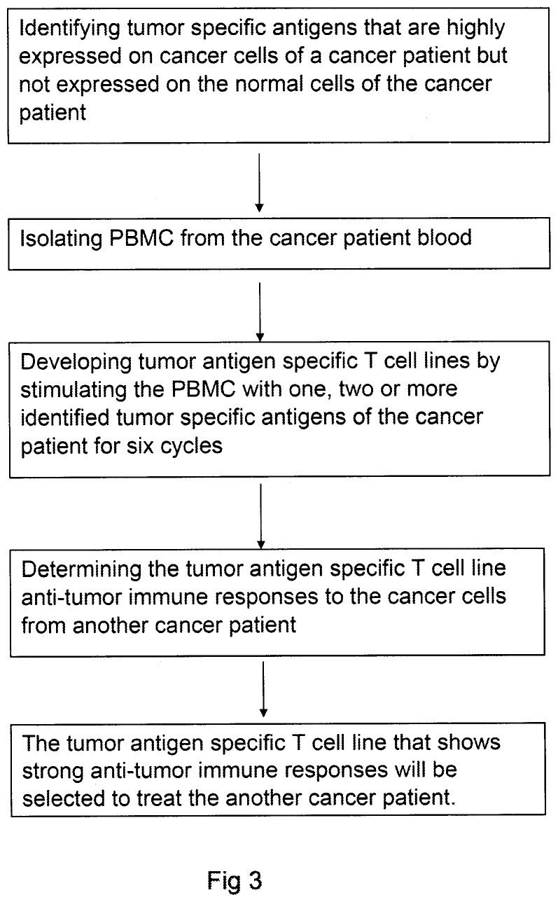

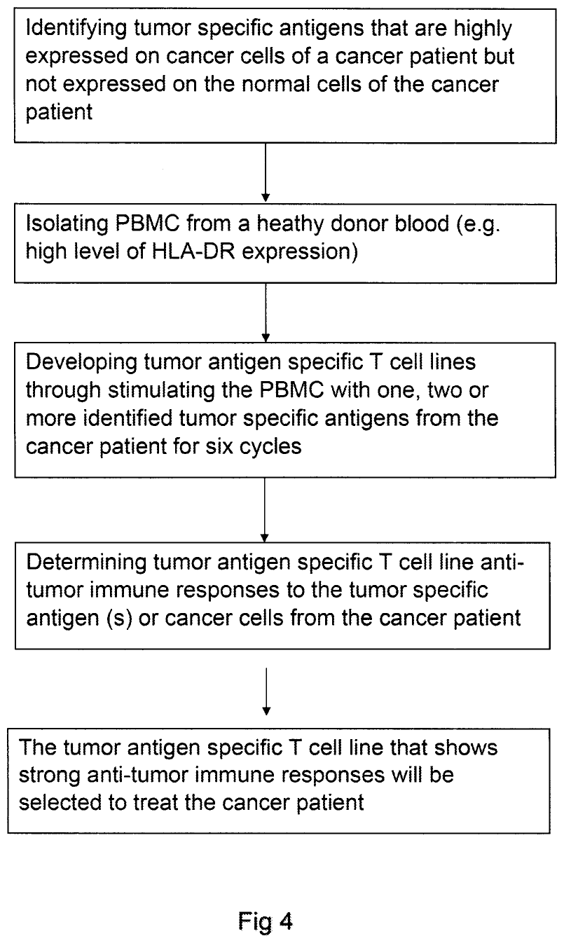

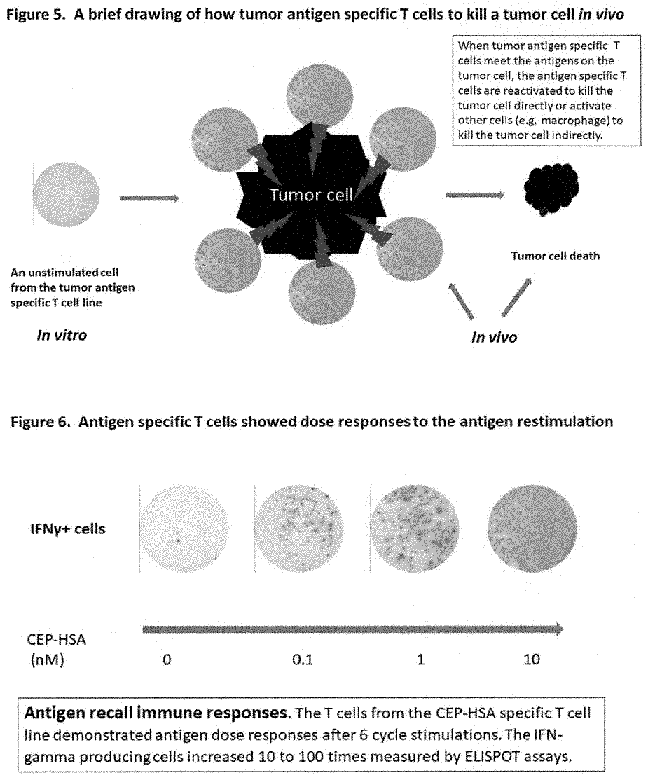

### DETAILED DESCRIPTION OF INVENTION

The present invention relates to the immunotherapy of cancer. The present invention relates to develop personalized tumor specific antigen tumor T cell lines to treat all types of cancer. Tumor antigen specific T cell lines develop in vitro using a cancer patient's peripheral blood mononuclear cells (PBMC) or an identified healthy donor PBMC. The tumor antigen specific T cell line that has strong anti-tumor immune responses will be selected to treat the cancer patient to cure the cancer. Personalized tumor antigen specific T cell lines are a safe and effective immunotherapy for cancer treatment. It provides a promise that cancer will be no longer an incurable disease.

How to produce a large number of effective tumor antigen specific T cells that are available for a long-term use is extremely important for curing cancer.

The present invention provides a method of generating tumor antigen specific T cell lines that provide potent and abundant tumor antigen specific T cells. The tumor antigen specific T cell lines are able to maintain in a cell culture incubator for two years or longer without loss of their anti-tumor immunity. In addition, the tumor antigen specific T cell lines can be frozen and stored in a liquid nitrogen freezer, thawed and expanded in a cell culture incubator again as needed.

The personalized tumor antigen specific T cell lines selectively recognize cancer cells to eliminate the cancer cells. This method can treat all types of cancer, comprising head and neck cancer, eye cancer (e.g. retinoblastoma, melanoma), brain tumors (e.g. glioblastoma), lung cancer, gastrointestinal system cancer (e.g. colon cancer, gastric cancer, liver cancer, pancreatic cancer, esophageal cancer), breast cancer, urology system cancer (e. g. kidney cancer, urinary bladder cancer), female reproductive system cancer (e.g. ovarian cancer, uterine cancer, cervical cancer), male system cancer (e.g. prostate cancer), blood cancer (e.g. acute and chronic leukemia, lymphoma), skin cancer (e.g. melanoma), mesothelioma, and endocrine cancer (e.g. thyroid cancer).
The detail method (step by step) is listed as follows.

1. Identify tumor specific antigens that highly express on tumor cells of a cancer patient but do not express on normal cells of the cancer patient by screening the cancer patient's tumor cells and his/her normal cells using available methods comprising next generation sequencing, RNA-Seq, western blots, proteomics, mass spectrometry, MHC binding capacity assessment, T cell epitope identification and sequence on human class I/II MHC molecules, and/or artificial intelligence.

2. The tumor specific antigens include tumor specific proteins, tumor specific peptides or other tumor specific molecules.

3. If a tumor specific antigen is a small molecule, it may have to conjugate with a low immunogenicity normal human protein such as human serum albumin.

4. While waiting for the identification results of tumor specific antigens (e.g. proteins, peptides) of a cancer patient, the tumor cells from the biopsy or surgery of the cancer patient are treated with mitomycin as a quick source of tumor specific antigens to develop a first tumor antigen specific T cell line.

5. Take blood from the cancer patient and isolate PBMC.

6. Generate a tumor antigen specific T cell line by incubating a cancer patient own PBMC with mitomycin-treated autologous tumor cells for six cycles or more; Each cycle is 5 days to 10 days; and the cell culture medium will be changed with fresh T cell medium containing mitomycin -treated tumor cells and IL2 (10 ng/ml) at the end of each cycle.

7. Culture tumor cells in a cell culture incubator to provide the source of tumor cells continuously.

8. Develop and expand tumor antigen specific T cell lines by stimulating a cancer patient own PBMC with one, two or more identified tumor specific antigens (e.g. proteins, peptides) from the cancer patient for six cycles or more cycles; Each cycle is 5 days to 10 days; the cell culture medium will be changed with fresh T cell medium containing the tumor specific antigen(s) and IL2 (10 ng/ml) at the end of each cycle.

9. The tumor antigen specific T cell line that shows strong anti-tumor immune responses to the tumor specific antigen(s) or tumor cells of a cancer patient is selected to treat the cancer patient.

10. T cells from one or more tumor antigen specific T cell lines can be administered to the cancer patient if one or more tumor antigen specific T cell lines show strong anti-tumor immune responses.

11. Tumor antigen specific T cell lines are developed and expanded from an identified healthy donor PBMC (e.g. high expression of HLA-DR) stimulated with one, two or more tumor specific antigens (e.g. proteins, peptides) from a cancer patient for six cycles or more. The tumor antigen specific T cell line that shows strong anti-tumor immune responses to the tumor specific antigen(s) or cancer cells from the cancer patient will be selected to treat the cancer patient.

12. A tumor antigen specific T cell line that is developed from a cancer patient's PBMC stimulated with the cancer patient's tumor specific antigen(s) shows strong anti-tumor immune responses to the cancer cells from another cancer patient and the tumor specific T cell line also can be used to treat the another cancer patient.

13. The tumor antigen specific T cell lines are maintained and expanded in a cell culture incubator (37° C. and 5% CO2) for two years or longer without losing their effective anti-tumor T immunity.

14. The tumor antigen specific T cell lines can be frozen and stored in a liquid nitrogen freezer, thawed and expanded in a cell culture incubator as needed and do not lose their effective anti-tumor T cell immunity as well.

15. The tumor antigen specific T cell line comprises CD8 positive T cells and/or CD4-positive T cells. The tumor antigen specific CD8-positive cytotoxic T cells can selectively recognize cancer cells and reactivate to kill the cancer cells directly. The tumor antigen specific CD4-positive T cells release IFN-g, TNF-alpha and other cytokines, support cytotoxic T cell function and anti-tumor antibody production, and attract other effector cells (e.g. natural killer cells, macrophages, dendritic cells and granulocytes) to the tumor site to kill the cancer cells.

16. The T cells from one or more tumor antigen specific T cell lines can be administered to a cancer patient simultaneously.

17. The T cells from the tumor antigen specific T cell line (s) can be administered to a cancer patient multiple times as needed.

18. This method can treat a cancer patient alone or in the combination of other cancer therapies such as surgery, radiation, chemotherapy, and/or other cancer immunotherapies (e.g. anti-PD-1 antibody).

19. The method of generating tumor antigen specific T cell lines works for non-solid cancer as well, comprising a) identify cancer specific antigens (proteins, peptides) from the cancer cells of a blood cancer patient; b) develop cancer specific T cell lines in vitro using PBMC from an identified healthy donor; c) stimulating the PBMC with the identified cancer specific antigens for six cycles or more; d) testing the anti-cancer immune responses of the cancer antigen specific T cell lines; and e) The cancer antigen specific T cell line that has strong anti-cancer immune responses will be selected to treat the blood cancer patient.

### EXAMPLE

### Methods

Peripheral blood mononuclear cells (PBMC) were isolated from twenty patients who had age-related macular degeneration (AMD) and twenty matched healthy donors. PBMC were stimulated with carboxyethylpyrrole-conjugated human serum albumin (CEP-HAS) and IL-2 for six cycles. Each cycle was five to ten days. At the end of each cycle, the cell culture medium was changed with fresh T cell medium containing CEP-HAS and IL-2 at 10 ng/ml.

### Results

The CEP-HAS antigen specific T cell lines demonstrated dose responses to CEP-HAS re-stimulation after 6 cycle stimulations. IFN-gamma producing cells increased about 10 to 100 times (FIG. 6). The same results were obtained with several other different human proteins and peptides. This method definitely works well for tumor specific antigens. Those antigen specific T cell lines had been maintained in a cell culture incubator for more than two years and did not lose their anti-antigen effector T cell immunity (e.g. 10 to 100 times increased IFN-gamma producing cells to the antigen re-stimulation). The antigen specific T cell lines were frozen and stored in a liquid nitrogen freezer for many years. After many years, the T cells from those antigen specific T cell lines were thawed and expanded in a cell culture incubator. Their effector T cell immune responses (e.g. IFN-g production) to the antigen re-stimulation were still as strong as before freezing and storing in the liquid nitrogen freezer.

1. A method of generating personalized tumor antigen specific T cell lines for cancer treatment, comprising tumor antigen specific T cell lines are generated by using peripheral blood mononuclear cells (PBMC) repeatedly stimulated in vitro with one, two or more tumor specific antigens for six cycles or more, wherein the tumor antigen specific T cell lines produce abundant and potent tumor antigen specific T cells that selectively recognize cancer cells and destroy the cancer cells, wherein the tumor antigen specific T cell lines are available for long-term use.

2. The method of claim 1, wherein the personalized tumor antigen specific T cell lines are used in the treatment of to treat all types of cancer, wherein the cancer is selected from the group comprising head and neck cancer, eye cancer, brain tumors, lung cancer, gastrointestinal cancer, breast cancer, urology system cancer, female reproductive system cancer, male reproductive system cancer, blood cancer, skin cancer, mesothelioma, and endocrine cancer.

3. The method of claim 2, wherein the head and neck cancer is selected from the group comprising laryngeal and hypo pharyngeal cancer, nasopharyngeal cancer, oropharyngeal cancer and salivary gland cancer, wherein the eye cancer is selected from the group comprising retinoblastoma and ocular melanoma, wherein the brain tumors are selected from the group comprising glioblastoma, astrocytoma and oligodendroglioma, wherein the gastrointestinal cancer is selected from the group comprising colon cancer, gastric cancer, liver cancer, pancreatic cancer, cholangiocarcinoma, gallbladder cancer and esophageal cancer, wherein the urology system cancer is selected from the group comprising kidney cancer, urinary tract cancer and bladder cancer, wherein the female reproductive system cancer is selected from the group comprising ovarian cancer, uterine cancer and cervical cancer, wherein the male reproductive system cancer is selected from the group comprising prostate cancer and testicular cancer, wherein the blood cancer is selected from the group comprising leukemia, lymphoma and multiple myeloma, wherein the skin cancer is selected from the group comprising melanoma, basal cell carcinoma and squamous carcinoma, and wherein the endocrine cancer is selected from the group comprising thyroid cancer, parathyroid cancer and neuroblastoma.

4. The method of claim 1, wherein each cycle comprises five days to ten days, and T cell medium (containing the tumor specific antigen(s) and IL-2 refreshed at the end of each cycle.

5. The method of claim 1, wherein the tumor antigen specific T cell lines are maintained and expanded in a cell culture incubator for two years or longer without loss of their anti-tumor T cell immunity.

6. The method of claim 1, wherein the tumor antigen specific T cell lines are frozen and stored in a liquid nitrogen freezer, thawed and expanded in a cell culture incubator again as needed.

7. The method of claim 1, wherein the tumor specific antigens comprise proteins, peptides, polysaccharides, lipids, small molecules or mitomycin-treated tumor cells, wherein the tumor specific antigens such as lipids or small molecules are conjugated with low immunogenicity human proteins, said human serum albumin to increase anti-tumor immunity.

8. The method of claim 1, wherein the tumor specific antigens that are expressed on tumor cells of a cancer patient but not expressed or limitedly expressed on normal cells of the cancer patient.

9. The method of claim 8, wherein the tumor specific antigen identification is performed on each individual cancer patient by methods comprising next generation sequencing, RNA-Seq, western blots, proteomics, mass spectrometry, MHC binding capacity assessment, T cell epitope identification and peptide sequence expression on human class I/II MHC molecules and/or artificial intelligence (AI).

10. The method of claim 1, wherein the tumor antigen specific T cell line is autologous or allogeneic to patients.

11. The method of claim 1, further comprising evaluating the anti-tumor immune responses of the tumor antigen specific T cell lines by methods comprising ELISA, ELISPOTs, flow cytometry, immunohistochemistry and/or in vivo testing.

12. The method of claim 11, wherein the tumor antigen specific T cell line that demonstrates anti-tumor immune responses to the tumor specific antigen(s) or cancer cells is selected for treatment.

13. The method of claim 1, wherein the population of the tumor antigen specific T cell lines comprise CD4 positive T cells or/and CD8-positive T cells.

14. The method of claim 2, wherein the personalized tumor antigen specific T cell line is an individual cancer immune therapy.

15. The method of claim 2, wherein the cancers are solid tumors or liquid tumors.

16. The method of claim 2, wherein the cancer is at its early stages or late stages.

17. The method of claim 1, wherein the T cells from one, two or more tumor antigen specific T cell lines are administered to a cancer patient.

18. The method of claim 17, wherein the T cells from the tumor antigen specific T cell line(s) are administered to a cancer patient multiple times as needed.

19. The method of claim 1, wherein the tumor antigen specific T cell line is used for cancer treatment alone or in combination with other cancer treatment such as surgery, radiation, chemotherapy, and/or other cancer immunotherapy.
